(19) United States
(12) Patent Application Publication
(10) Pub. No.: US 2023/0416721 A1
(43) Pub. Date: Dec. 28, 2023

(54) COMBINATION THERAPEUTICS USING TUMOR TREATING FIELDS (TTFIELDS)

(71) Applicant: THE BOARD OF REGENTS OF THE UNIVERSITY OF TEXAS SYSTEM, Austin, TX (US)

(72) Inventors: Michael D. STORY, Dallas, TX (US); Narasimha Kumar KARANAM, Dallas, TX (US)

(73) Assignee: THE BOARD OF REGENTS OF THE UNIVERSITY OF TEXAS SYSTEM, Austin, TX (US)

(21) Appl. No.: 18/220,864

(22) Filed: Jul. 12, 2023

Related U.S. Application Data
(63) Continuation of application No. 17/208,107, filed on Mar. 22, 2021, now abandoned.
(60) Provisional application No. 62/993,603, filed on Mar. 23, 2020.

(51) Int. Cl. C12N 13/00; A61P 35/00; A61K 45/06

(52) U.S. Cl. CPC C12N 13/00; A61P 35/00; A61K 45/06

(57) ABSTRACT

Methods of reducing survival of cancer cells in a subject by applying alternating electric fields to the cancer cells and delivering at least one of an E2F inhibitor and a CDK4/6 inhibitor to the cancer cells are provided. In some instances, alternating electric fields are applied to the cancer cells at a frequency between 80 and 300 kHz. In some instances, at least a portion of the applying step is performed simultaneously with at least a portion of the delivering step. In some aspects, the alternating electric fields are applied to the cancer cells for at least 72 hours.

### CROSS REFERENCE TO RELATED APPLICATIONS

This application claims the benefit of U.S. Provisional Application 62/993,603, filed Mar. 23, 2020, which is incorporated herein by reference in its entirety.

All references cited herein, including but not limited to patents and patent applications, are incorporated by reference in their entirety

### BACKGROUND

Lung cancer is the second most prevalent cancer and the leading cause of cancer-related death in the United States. Non-small cell lung cancer (NSCLC) is the most prevalent type, accounting for approximately 80% of new cases. A plethora of treatment options for lung cancer exist including surgical resection, chemotherapy, radiation therapy, and immunotherapy. Five-year survival rates for patients with stage I and II NSCLC are about 50% and 30%, respectively. However, despite the available treatment options, 5-year survival rates for patients with late stage IIIA, IIIB and IV are 14%, 5% and 1%, respectively, highlighting the need for novel treatment modalities that can be utilized alone or in combination with conventional therapies to increase survival rates.

In addition, many cancers, including lung cancer, are associated with defects in genes that function in DNA repair mechanisms. These genes are members of gene regulatory pathways associated with development of various cancers (e.g., BRCA pathway, Fanconi anemia (FA)BRCA pathway).

TTFields are a non-invasive physical modality of cancer therapy that is approved for recurrent and newly diagnosed glioblastoma multiforme (GBM) in combination with temozolomide, and unresectable locally advanced or metastatic malignant pleural mesothelioma (MPM) in combination with platinum based chemotherapy. Clinical trials are ongoing for other cancers, including lung, pancreatic, and ovarian cancers.

### SUMMARY

The present disclosure provides enhancements and improvements to existing treatment mechanisms that utilize TTFields, by for example, combining treatment using TTFields with a E2F inhibitor (e.g., HLM006474) and/or a CDK 4/6 inhibitor (e.g., abemaciclib). Embodiments of the present disclosure target specific aspects of the CDK-RB-E2F signal pathways. Embodiments of the present disclosure selectively downregulate and upregulate aspects of the CDK-RB-E2F signal pathways. Aspects described herein combine TTFields with agents that target cancers through reductions in DNA repair capacity via multiple pathways that rely on specific DNA repair pathways (e.g., homologous recombination, non-homologous end joining, mis-match repair, replication fork maintenance and chromosome maintenance, amongst others). E2F is a ubiquitous transcription factor involved in cell cycle control, DNA repair, and chromosomal maintenance routines in every cell type. By combining TTFields with, for example, E2F inhibitors, aspects described herein target a wide range of cancers and cell types.

Aspects described herein provide methods of reducing survival of cancer cells in a subject, by delivering at least one of an E2F inhibitor and a CDK4/6 inhibitor to the cancer cells and applying alternating electric fields to the cancer cells at a frequency between 80 and 300 kHz.

Aspects described herein provide methods of killing cancer cells in a subject by delivering at least one of an E2F inhibitor and a CDK4/6 inhibitor to the cancer cells and applying alternating electric fields to the cancer cells at a frequency between 80 and 300 kHz.

### BRIEF DESCRIPTION OF THE DRAWINGS

FIG. 1 illustrates conserved domains present in E2F proteins;

FIG. 2 illustrates an exemplary signal pathway of the proteome following exposure to TTFields;

FIG. 3A shows exemplary changes in the expression levels of the indicated E2F target following exposure to TTFields;

FIG. 3B is a continuation of FIG. 3A and shows exemplary changes in the expression levels of the indicated E2F target following exposure to TTFields;

FIG. 3C is a continuation of FIG. 3B and shows exemplary changes in the expression levels of the indicated E2F target following exposure to TTFields;

FIG. 3D is a continuation of FIG. 3C and shows exemplary changes in the expression levels of the indicated E2F target following exposure to TTFields;

FIG. 4 provides exemplary gene signature markers of E2F-RB dysfunction;

FIG. 5 provides an exemplary diagrammatic representation of the effects of combining TTFields with E2F inhibitors and CDK 4/6 inhibitors in accordance with aspects described herein;

FIG. 6A provides the results of exemplary clonogenic survival assays in four cell lines with the indicated combinations of TTFields, E2F inhibitors and CDK 4/6 inhibitors in accordance with aspects described herein;

FIG. 6B is a continuation of FIG. 6A and provides the results of exemplary clonogenic survival assays in four cell lines with the indicated combinations of TTFields, E2F inhibitors and CDK 4/6 inhibitors in accordance with aspects described herein;

FIG. 7A provides combination index values from the exemplary clonogenic assays of FIGS. 6A-6B, in accordance with some embodiments of the present disclosure;

FIG. 7B is a continuation of FIG. 7A and provides combination index values from the exemplary clonogenic assays of FIGS. 6A-6B, in accordance with some embodiments of the present disclosure;

FIG. 8A illustrates mRNA expression level over time in the indicated cell lines and protein expression levels for Fanconi anemia/BRCA pathway genes;

FIG. 8B is a continuation of FIG. 8A and illustrates mRNA expression level over time in the indicated cell lines and protein expression levels for Fanconi anemia/BRCA pathway genes;

FIG. 8C is a continuation of FIG. 8B and illustrates mRNA expression level over time in the indicated cell lines and protein expression levels for Fanconi anemia/BRCA pathway genes;

FIG. 8D is a continuation of FIG. 8C and illustrates mRNA expression level over time in the indicated cell lines and protein expression levels for Fanconi anemia/BRCA pathway genes;

FIG. 8E is a continuation of FIG. 8D and illustrates mRNA expression level over time in the indicated cell lines and protein expression levels for Fanconi anemia/BRCA pathway genes;

FIG. 8F is a continuation of FIG. 8E and illustrates mRNA expression level over time in the indicated cell lines and protein expression levels for Fanconi anemia/BRCA pathway genes;

FIG. 9A illustrates transcriptional activity and gene expression of FA pathway genes, cell cycle genes, and DNA replication genes following exposure of cancer cells to TTFields;

FIG. 9B is a continuation of FIG. 9A and illustrates transcriptional activity and gene expression of FA pathway genes, cell cycle genes, and DNA replication genes following exposure of cancer cells to TTFields; and

FIG. 10 provides exemplary combination index values resulting from clonogenic assays for indicated combinations of TTFields and various drugs at 24, 48, and 72 hours after exposure to TTFields, including E2F and CDK inhibitors, in H1299 cells, in accordance with aspects described herein.

### DETAILED DESCRIPTION

The present disclosure relates to the application of TTFields in combination with therapeutic agents in order to target pathways associated with cancer cell growth and viability.

Embodiments of the present disclosure are directed to the application of TTFields and delivery of therapeutic agents to cancer cells, in order to dysregulate members of the E2F family known to play an active role in cancer cell replication. For example, the disclosed combination therapeutics using TTFields combines TTFields with E2F and/or CDK 4/6 inhibitors to downregulate E2F1 and E2F2 of the CDK-RB-E2F axis. Additionally, in some embodiments, a combined therapeutic application combines TTFields with E2F and/or CDK 4/6 inhibitors to upregulate E2F6 of the CDK-RB-E2F axis.

TTFields dysregulate the E2F family of transcription factors and render tumor cells susceptible to agents targeting the RB-E2F-CDK4/6 axis, thus substantially increasing the tumoricidal effects of TTFields alone or with agents that target DNA repair, replication stress, and other pathways regulated by the RB-E2F-CDK4/6 axis.

TTFields are used for recurrent and newly diagnosed glioblastoma multiformae (GBM) and pleural mesothelioma. TTFields are used to induce cell death via disruption of mitosis, adding replication stress and down-regulating DNA repair and cell cycle checkpoint genes.

TTFields are used for the treatment of solid, therapy-resistant primary and recurrent tumors. TTFields electrodes are non-invasive and deliver a low-intensity (e.g., 1-3 V/cm) intermediate frequency (e.g., 100-300 kHz) alternating electric field across the tumor bed. TTFields create a heterogeneous intracellular environment that induces a dielectrophoretic movement of polar molecules toward the region of higher field intensity, effectively preventing polymerization and other critical biochemical functions. As such, TTFields preferentially target cancer cells through the exploitation of cell proliferation, effectively sparing non-dividing normal cells. In addition, TTFields do not stimulate nerves and muscle because of their high frequency, and do not generate high levels of heating because of their low intensity.

TTFields elicit a conditional vulnerability to ionizing radiation (IR) in Non-Small Cell Lung Cancer Cell (NSCL Cell Lines). TTFields induce a state of conditional susceptibility resulting in enhanced sensitivity to ionizing radiation and supports the use of TTFields as a combined modality therapy with radiation, PARP inhibitors, other DNA-damaging agents, E2F inhibitors, and CDK 4/6 inhibitors.

TTFields decrease cellular proliferation and induce abortive apoptosis in dividing cancer cells across a variety of human and rodent tumor cell lines. Prevention of proper formation of the mitotic spindle apparatus and the activation of the mitotic spindle checkpoint has been proposed as the mechanism by which TTFields kill dividing cells. Specifically, TTFields exposure leads to microtubule depolymerization and the mislocalization of septin. This can result in plasma membrane instability and blebbing that disrupts cytokinesis, leading to abnormal chromosome segregation, aberrant mitotic exit and production of deranged cells that subsequently undergo apoptosis.

Aspects described herein provide methods of reducing survival of cancer cells in a subject, by delivering at least one of an E2F inhibitor and a CDK4/6 inhibitor to the cancer cells and applying alternating electric fields to the cancer cells at a frequency between 80 and 300 kHz.

In some instances, at least a portion of the applying step is performed simultaneously with at least a portion of the delivering step. In some instances, the applying step has a duration of at least 72 hours. In another instance, the applying step has a duration of at least 24 hours or 48 hours.

In some instances, the frequency of the alternating electric field is between 100 and 200 kHz. In another instance, the alternating electric field has a field strength of at least 1 V/cm in at least some of the cancer cells.

The concentration of the E2F inhibitor in the cancer cells can be from about 10 μM to about 50 μM or about 20 μM to about 40 μM. The concentration of the CDK4/6 inhibitor in the cancer cells can be from about 0.1 μM to about 5 μM or about 0.5 μM to about 2 μM.

In some instances, IC25 values for a CDK inhibitor (e.g., HLM 006474) can be about 0.5 μM, about 1 μM, and about 1.5 μM. In another aspect, about 0.5 to about 1.5 μM can be used for the concentration of the CDK inhibitor in the cancer cells.

In some instances, IC25 values for E2F inhibitor (e.g., LY2835219 aka abemaciclib) can be about 20 μM, about 25 μM, or about 40 μM. In another aspect, the concentration of the E2F inhibitor in the cancer cells can be from about 20 μM to about 40 μM.

In yet another instance, the E2F inhibitor is selected from the group consisting of one or more of HLM006474, MRT00033659, YKL-5-124-TFA, and YKL-5-124.

In a further instance the CDK4/6 inhibitor is selected from the group consisting of one or more of abemaciclib, ribociclib, trilaciclib, ibrance, lerociclib, alvocidib, roniciclib, riviciclib, milciclib, RGB-286638, NSN3106729, PHA-793887, R547, indirubin, NU6102, bohemine, CDK9-IN-7, CGP60474, purvalanol A, PF-06873600, nimbolide, FN-1501, AG-024322, ON123300, G1T28, G1T38, AMG925, SHR-6390, BPI-1178, BPI-16350, FCN437, birociclib, BEBT-209, Ty-302, TQB-3616, HS-10342, PF-06842874, CS-2002, MM-D37K, CDK4/6-IN-2, SU9516, and AT7519.

In some instances, the delivering step comprises administering or delivering the E2F inhibitor and the CDK4/6 inhibitor to the cancer cells. In some instances, the delivering step comprises administering or delivering the E2F inhibitor and the CDK4/6 inhibitor to the subject. In one aspect, the E2F inhibitor is HLM006474. In a further aspect, the CDK4/6 inhibitor is abemaciclib. In yet another instance, the E2F inhibitor is HLM006474, and the CDK4/6 inhibitor is abemaciclib.

In some instances, the cancer cells are selected from the group consisting of lung cancer cells, breast cancer cells, pancreatic cancer cells, glioblastoma cells, prostate cancer cells, liver cancer cells, fallopian tube cancer cells, peritoneal cancer cells, skin cancer cells, and ovarian cancer cells.

E2F transcription factors are active in every cell type and are associated with, for example, cell cycle control, DNA repair, and chromosomal maintenance routines. HLM 006474, an E2F inhibitor, inhibits DNA binding for all E2F complexes and has been used in breast cancer and melanoma models. CDK 4/6 inhibitors have been used to treat glioblastoma and metastatic breast cancer.

In some instances, the survival of the cancer cells is reduced by 20 to 100 fold compared to cancer cells that do not receive exposure to alternating electric fields and do not receive delivery of an E2F inhibitor and a CDK4/6 inhibitor.

In some instances, the survival of the cancer cells is reduced by about 100 fold after (i) 72 hours exposure of the cancer cells to alternating electric fields, (ii) delivery of the E2F inhibitor at a concentration of 10 μM to 50 and (iii) delivery of the CDK4/6 inhibitor at a concentration of 0.1 μM to 2 μM compared to cancer cells that do not receive exposure to alternating electric fields and do not receive delivery of an E2F inhibitor and a CDK4/6 inhibitor. In some instances, the E2F inhibitor is delivered to the cancer cells at a concentration of about 20 μM and the CDK4/6 inhibitor is delivered to the cancer cells at a concentration of about 5 μM.

Aspects described herein provide methods of killing cancer cells in a subject by delivering at least one of an E2F inhibitor and a CDK4/6 inhibitor to the cancer cells and applying alternating electric fields to the cancer cells at a frequency between 80 and 300 kHz.

In some instances, at least a portion of the applying step is performed simultaneously with at least a portion of the delivering step. The applying step can have a duration of at least 72 hours. In another aspect, the applying step can have a duration of at least 24 hours or 48 hours.

The E2F inhibitor can be selected from the group consisting of one or more of HLM006474, MRT00033659, YKL-5-124-TFA, and YKL-5-124.

The CDK4/6 inhibitor can be selected from the group consisting of one or more of abemaciclib, ribociclib, trilaciclib, ibrance, lerociclib, alvocidib, roniciclib, riviciclib, milciclib, RGB-286638, NSN3106729, PHA-793887, R547, indirubin, NU6102, bohemine, CDK9-IN-7, CGP60474, purvalanol A, PF-06873600, nimbolide, FN-1501, AG-024322, ON123300, G1T28, G1T38, AMG925, SHR-6390, BPI-1178, BPI-16350, FCN437, birociclib, BEBT-209, Ty-302, TQB-3616, HS-10342, PF-06842874, CS-2002, MM-D37K, CDK4/6-IN-2, SU9516, and AT7519.

In some instances, the E2F inhibitor is HLM006474. In some instances, the CDK4/6 inhibitor is abemaciclib. In yet another instance, the E2F inhibitor is HLM006474, and the CDK4/6 inhibitor is abemaciclib.

Experimental data presented below discusses the use of proteomic analysis to identify a mechanism by which TTFields and combination therapeutics are able to downregulate the key genes associated with cell cycle checkpoint and DNA repair and other survival pathways that present therapeutic challenges to conventional treatment systems.

As discussed herein, quantitative proteomics experiments identified reduced expression of E2F1 and E2F2, which are transcriptional activators. Additionally, quantitative proteomics experiments identified increased expression of E2F6, which are transcriptional repressors. Accordingly, the combination therapies described herein affect the CDK-RB-E2F axis and dysregulate DNA repair genes (e.g., RAD51, BRCA1, and BRCA2) through E2F4 and E2F6 signaling.

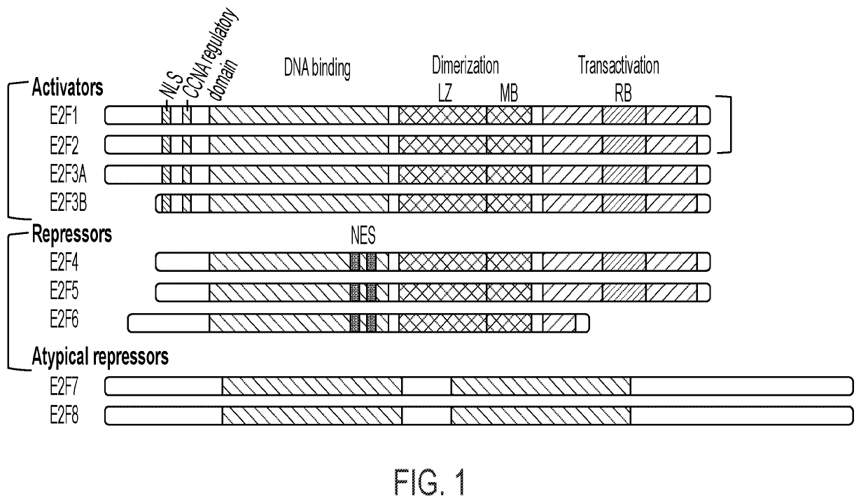

FIG. 1 provides an exemplary an overview of conserved domains (i.e., domains that are preserved and have a designated function) present in E2F proteins. E2F proteins consist of eight family members (E2F1-8), which, based on their function, are divided into transcriptional activators (E2F1-E2F3a) and transcriptional repressors (E2F3b-E2F8). E2F proteins regulate thousands of genes important for cell cycle progression, DNA replication, DNA damage checkpoint, and DNA repair, and plays a central role in cell proliferation. Activator protein (E2F1, E2F2 and E2F3A) levels peak at the G1-S phase transition, and atypical repressor (E2F7 and E2F8) levels peak later in late S phase, whereas canonical repressor (E2F3B, E2F4, E2F5 and E2F6) levels remain constitutively expressed throughout all phases of the cell cycle.

As illustrated, all E2Fs share a distinctive winged-helix DNA binding domain. E2F1, E2F2, E2F3, E2F4, E2F5 and E2F6 require dimerization with a member of the transcription factor dimerization partner (TFDP) family (TFDP1 or TFDP2) in order to bind DNA. This binding is facilitated by the dimerization domain, which consists of leucine zipper (LZ) and marked box (MB) domains. E2F1, E2F2, E2F3, E2F4 and E2F5 are bound by pocket proteins (RB, p107 and p130) at the transactivation domain.

The minimal site necessary for interaction with pocket proteins is shown (RB). E2F1, E2F2, E2F3, E2F4 and E2F5 are all bound by RB, while p107 and p130 only bind E2F4 and E2F5. E2F6 does not bind pocket proteins but instead is regulated by Polycomb group proteins. Some E2F proteins also have a nuclear localization sequence (NLS), a nuclear export sequence (NES) or cyclin A (CCNA) regulatory domains. E2F7 and E2F8 lack dimerization and transactivation domains and do not bind TFDPs or pocket proteins. Instead, they have two tandem DNA binding domains.

Exemplary upstream analysis of differentially expressed proteins upon exposure of cells to TTFields indicated that activators such as E2F1 and E2F2 are inhibited and repressors such as E2F6 are activated.

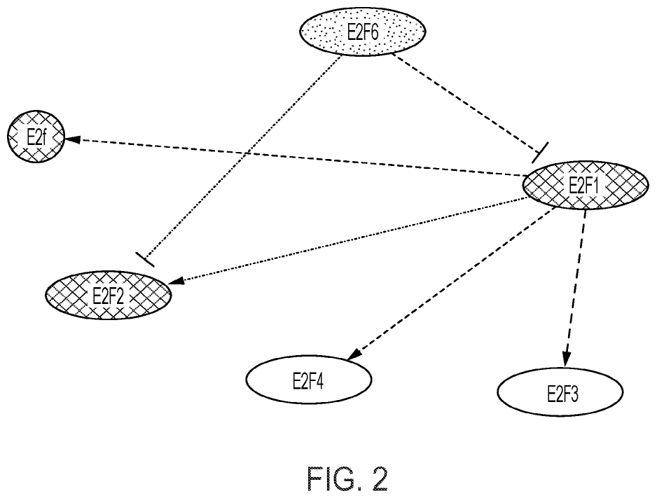

FIG. 2 provides an exemplary upstream analysis of the proteome following exposure of cells to TTFields. Proteomics results suggest that transcriptional activators (E2F1 and E2F2) are inhibited, and repressor (E2F6) is activated as a result of TTFields exposure.

The E2F-RB axis serves as a central junction controlling many pathways in cancer cells, including mitosis, DNA damage and repair, DNA replication, chromatin remodeling and apoptosis. Gene expression studies and direct functional studies have shown that the E2F-RB pathway targets several of the FA (Fanconi anemia) pathway members. Further, TTFields downregulate FA pathway members by dysregulation of E2F-RB (which acts as an upstream regulator of the FA pathway and cell cycle).

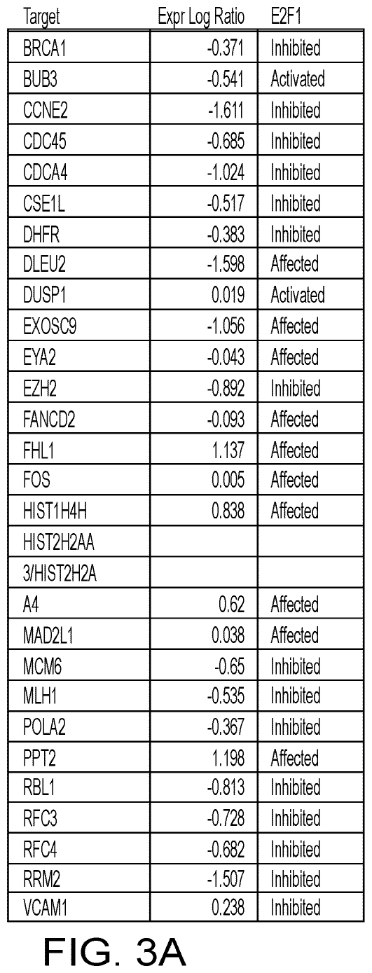

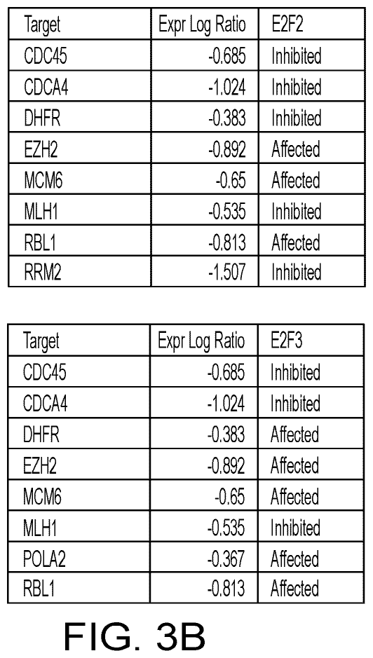

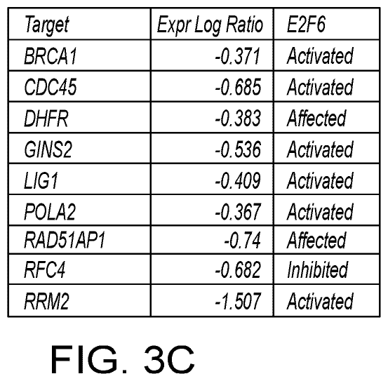

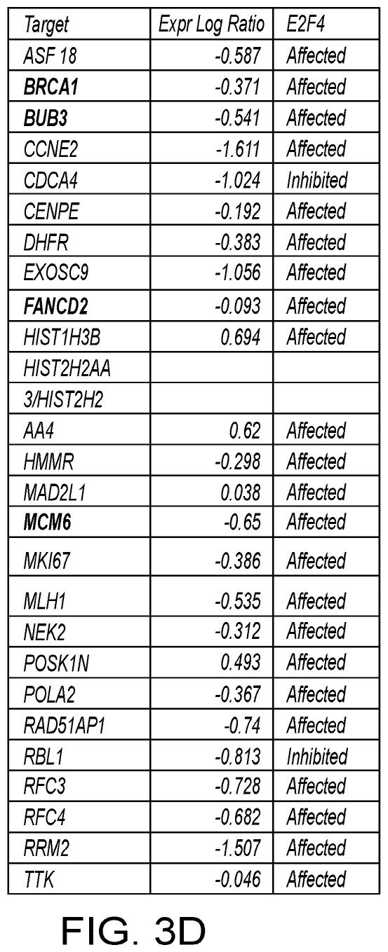

FIGS. 3A-3D provide exemplary results of an E2F target expression assay in lung cancer cells following exposure to TTFields compares to cells not exposed to TTFields based on a differential proteome analysis.

E2F targets examined in FIGS. 3A-3D include FA pathway members (e.g., BRCA1, FANCD2, RAD51), replication fork related members (e.g., MCM6, RFC3, RFC4), and mitosis related proteins (e.g., BUB3, CCNE2, EZH2). As illustrated in FIGS. 3A-3D, expression levels change upon exposure to TTFields as indicated by log expression values. Expression level changes were also categorized as inhibited, affected, or activated.

As shown in FIGS. 3A-3D, E2F1, E2F2 are activators and E2F4, E2F6 are inhibitors and were inhibited and activated respectively upon exposure to TTFields. Accordingly, the expression of their targets were decreased (e.g., BRCA1, MCM6, CCNE2, EZH2).

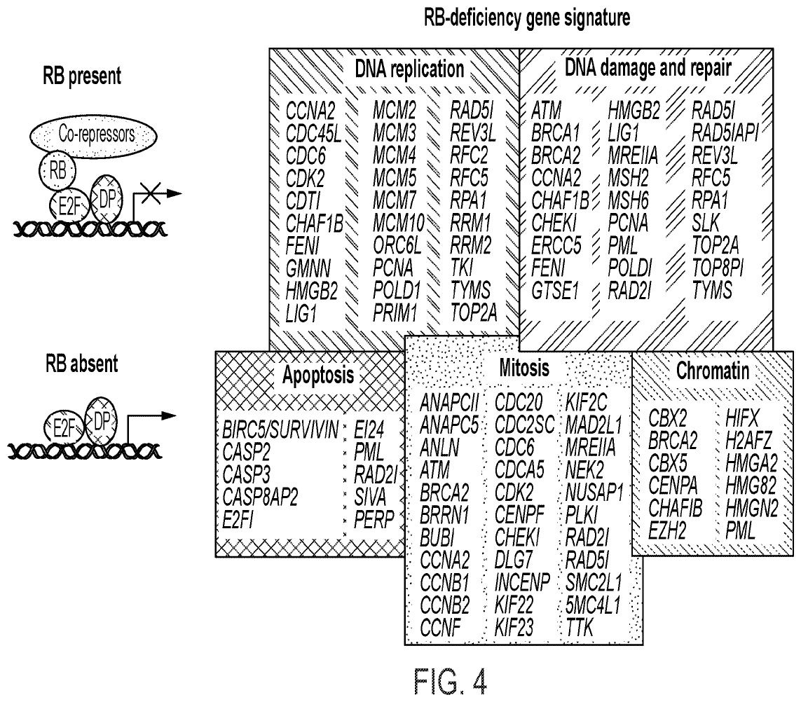

FIG. 4 provides exemplary gene signature markers of E2F-RB dysfunction. Gene-signature analyses have shown the functional groups of genes that are deregulated by the dysregulation/loss of E2F-RB. FIG. 4 identifies exemplary gene targets associated with DNA replication, DNA damage and repair, apoptosis, mitosis, and chromatin regions.

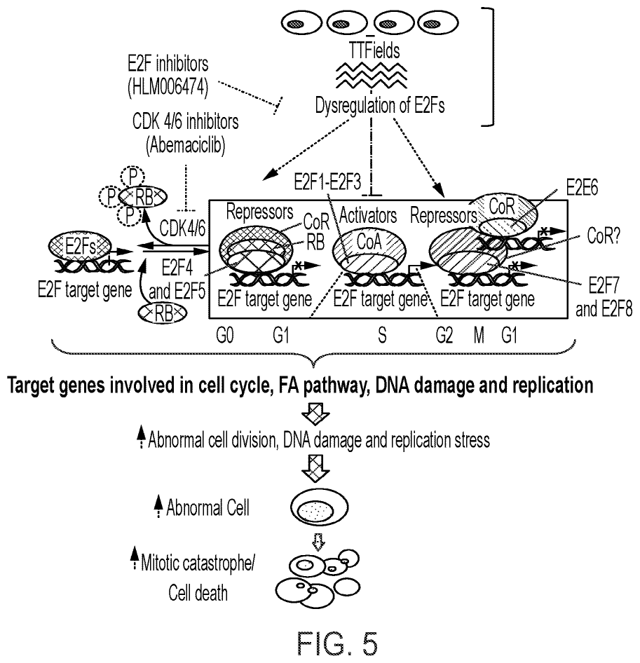

FIG. 5 provides a diagrammatic representation of exemplary combination therapies described herein with respect to TTFields, E2F inhibitors, and CDK 4/6 inhibitors. As illustrated, the combination of TTFields together with inhibitors of upstream regulators (E2Fs and CDK4/6) of cell cycle, DNA damage and replication stress which we observed as TTFields mechanisms of action would be highly beneficial. As disclosed herein, the E2F and CDK4/6 regulators can be targeted with E2F inhibitors (e.g., HLM006474) and CDK4/6 inhibitors (e.g., abemaciclib). Without being bound by theory, and as illustrated in FIG. 5, exposing cancer cells to TTFields and E2F inhibitors targets dysregulation of E2F gene targets while CDK 4/6 inhibitors target cell cycle progression. This combination can lead to abnormal cell division, DNA damage, replication stress, and ultimately cell death.

As illustrated in FIG. 5, TTFields can be applied to cancer cells in order to dysregulate the E2Fs. Additional E2F inhibitors (e.g., HLM006474) can be applied to the cancer cells to dysregulate E2F targets leading to an increase in expression of repressors decrease or inhibition of the expression of activators. CDK 4/6 inhibitors (e.g., abemaciclib) can also be applied inhibit CDK 4/6 activity and cell cycle expression and target genes involved in the cell cycle, FA, DNA damage, and replication leading to an increase in abnormal cell division, DNA damage and replication stress. These effects can lead to an increase in abnormal cells resulting in increased mitotic/catastrophic cancer cell death.

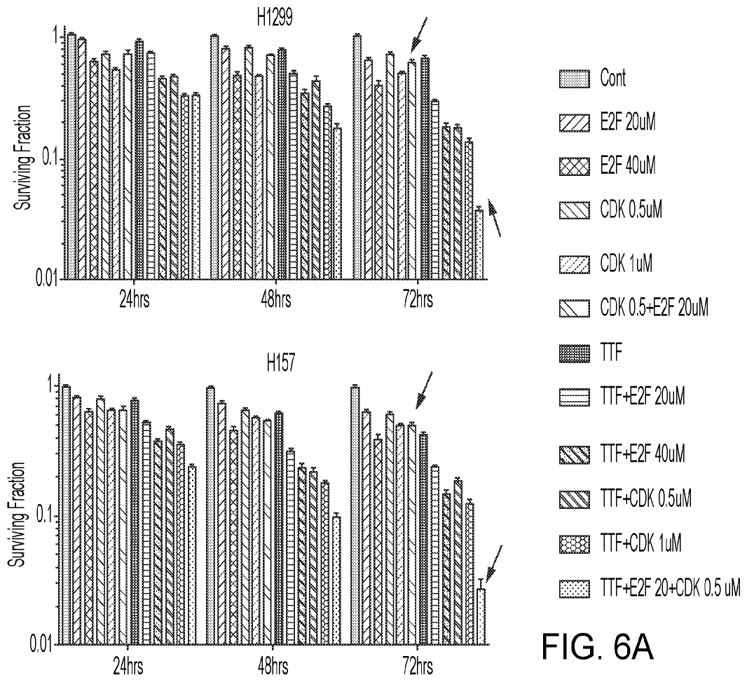

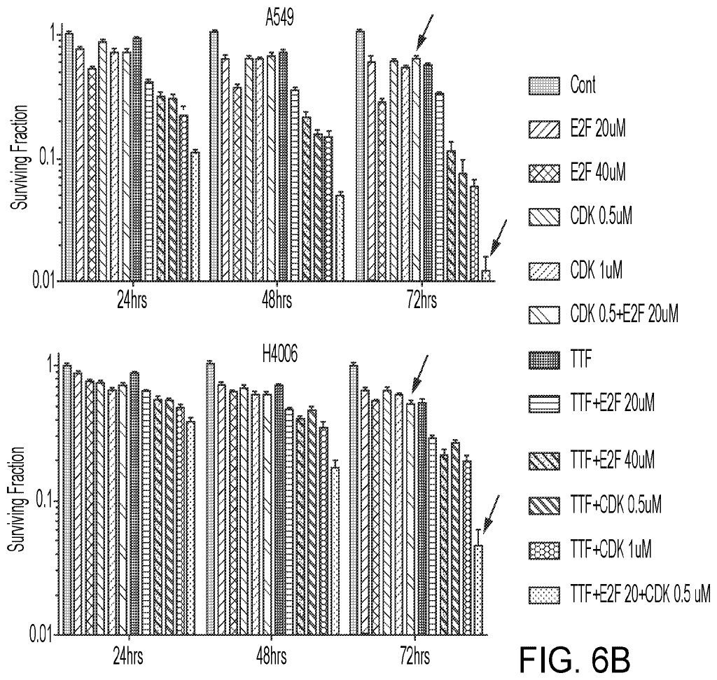

FIGS. 6A-6B provide the results of exemplary clonogenic survival assays in four lung cancer cells lines—H1299, A549, H157, and H4006 cells—under various conditions testing combinations of TTFields with agents that dysregulate E2F signaling or CDK4/6 signaling at 24, 48, and 72 hours. H1299 and A549 are non-small cell lung cancer cell lines that are resistant to conventional treatment using TTFields. By contrast H157 and H4006 are cell lines that are known to be more responsive to TTFields. Cell survival after increased exposure to TTFields alone, or in combination with a E2F inhibitor, a CDK 4/6 inhibitor or to a combination of E2F inhibitor and CDK 4/6 inhibitor is shown.

As shown in FIGS. 6A-6B, there is a 20 to 100 fold reduction in cell survival after seventy-two hours of TTFields exposure when lower doses of the E2F and CDK 4/6 inhibitors are used in combination with TTFields. In one example, the combination of TTFields exposure for 72 hours, 20 μM of E2F inhibitor HLM006474 and 0.5 μM of abemaciclib resulted in about a 100 fold reduction in cell survival. Without being bound by theory, it is believed that the unexpected reduction in cell survival resulted from targeting several pathways at once.

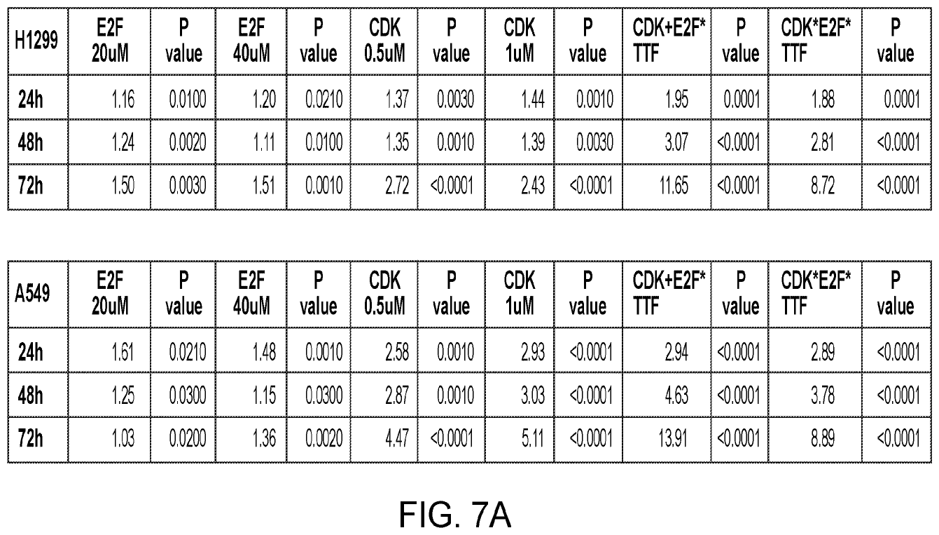

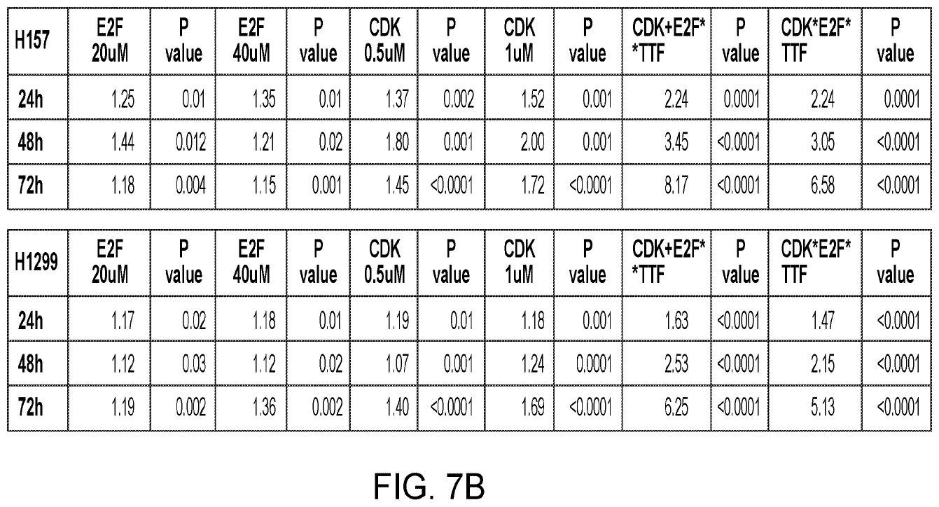

FIGS. 7A-7B provides combination index (CI) values from the exemplary clonogenic survival assays described in FIGS. 6A-6B. In some instances, CI values greater than 1.0 describe synergy, or surprisingly favorable effects for anti-tumor activity. As illustrated in FIGS. 7A-7B, H1299 and A549 cell lines show significant synergy 72 hours after TTFields are applied in combination with CDK+E2F inhibitors (e.g., CI index value of 11.65 in H1299 after 72 hours, and CI index value of 13.91 in A549 after 72 hours).

In some aspects, a combined therapeutic application combines the application of TTFields with an application or delivery of one or more therapeutic agents to cancer cells concurrently or immediately after application of the TTFields.

In some aspects, a combined therapeutic application combines the application of TTFields with an application of one or more therapeutic agents to cancer cells a pre-determined period of time after the application of the TTFields. The pre-determined period of time may be determined based on the observed vulnerability of the cancer cells after TTFields are applied. For example, in some aspects, the cancer cells can be treated or exposed to combination therapies approximately 24, 48, or 72 hours after TTFields are applied.

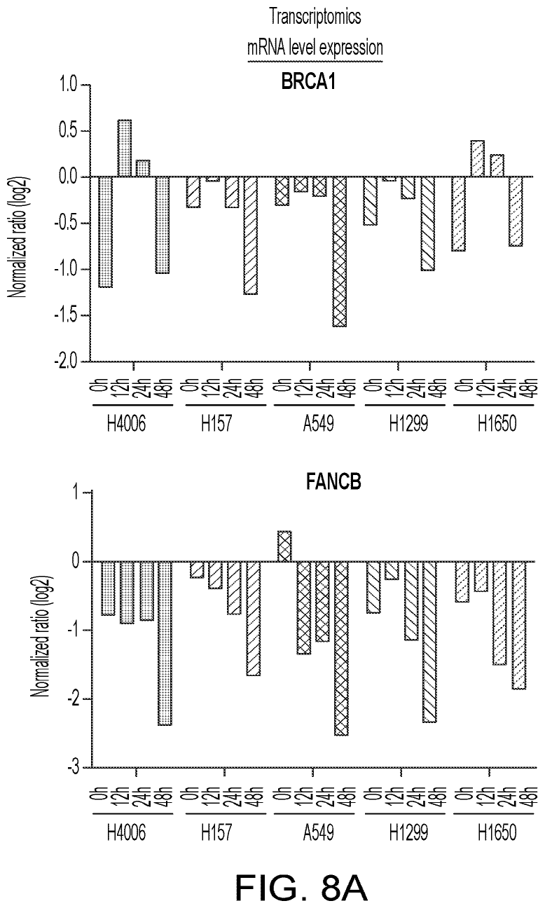

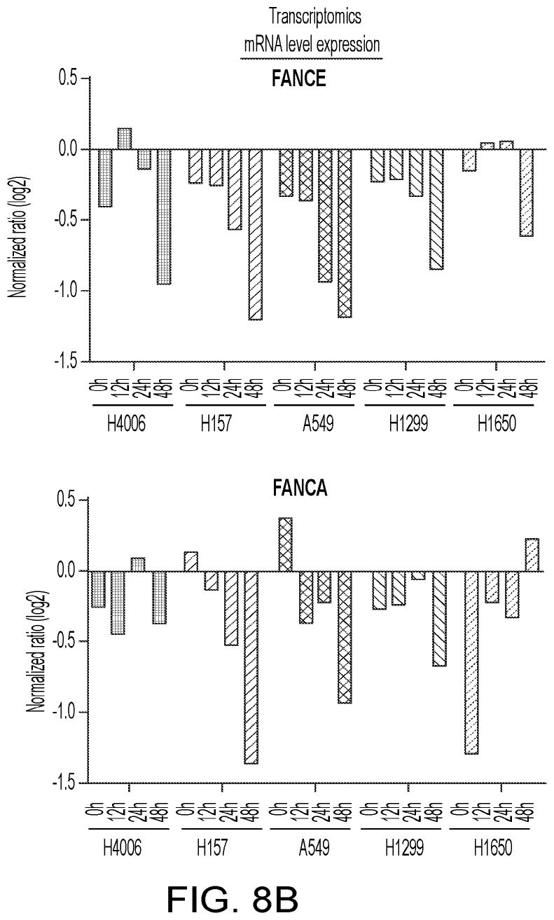

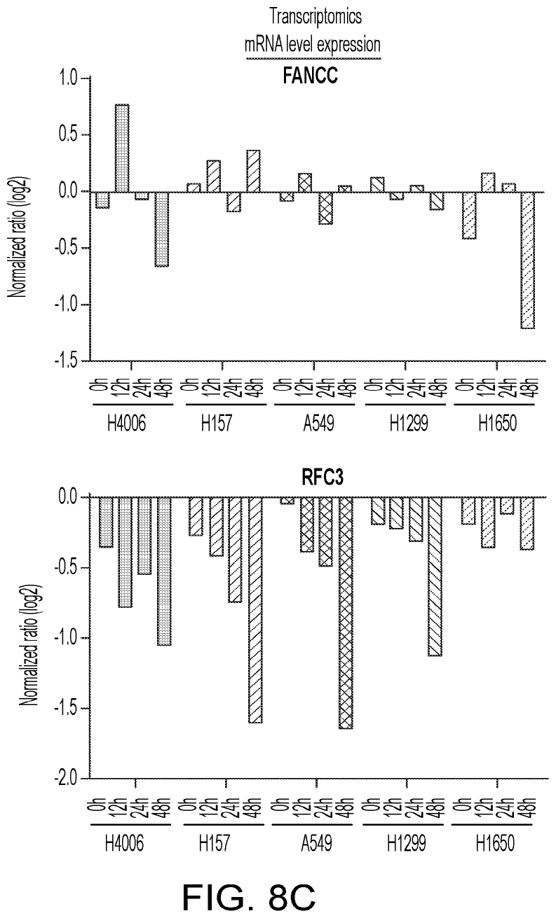

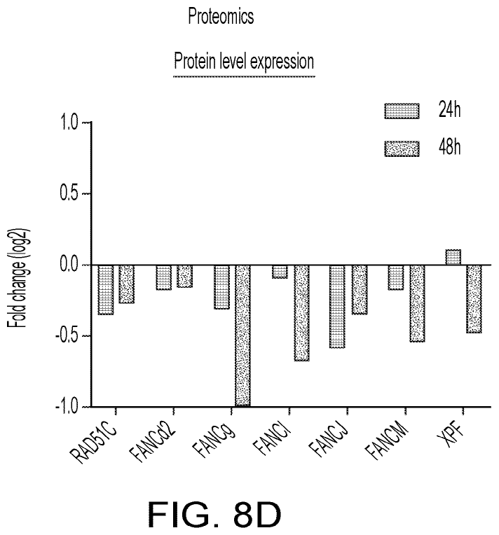

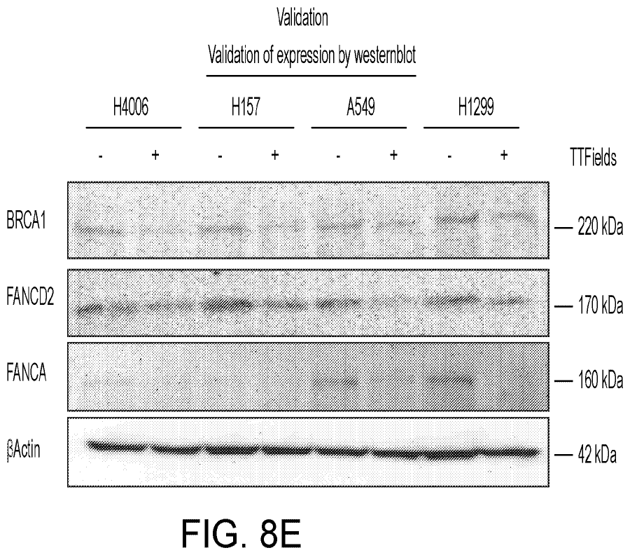

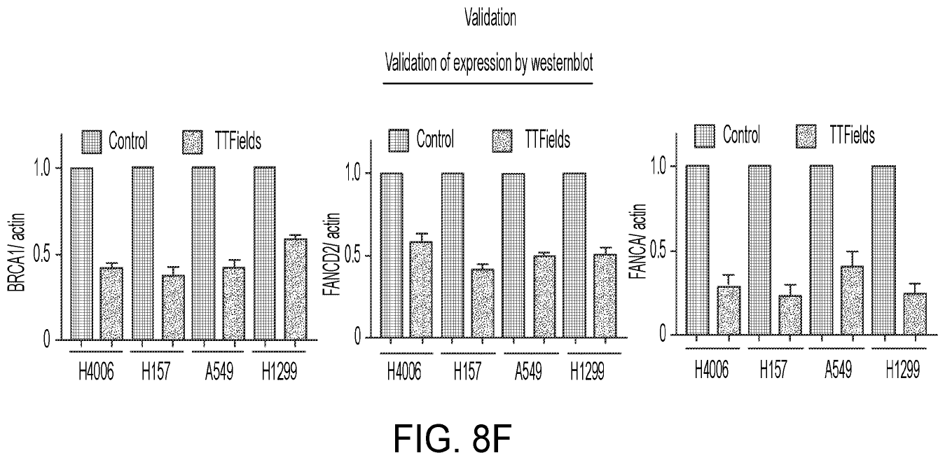

FIGS. 8A-8F provide exemplary data regarding mRNA and protein level expression for genes in the FA-BRACA Pathway (BRCA1, FANCE, FANCC, FANCB, FANCA, and RFC3) in H4006, H157, A549, H1299, and H1650 cells after exposure to TTFields for 24, 48, and 72 hours. As shown in FIGS. 8A-8F, TTFields decreases mRNA and protein expression for these genes with maximal decrease of expression shown after 72 hours.

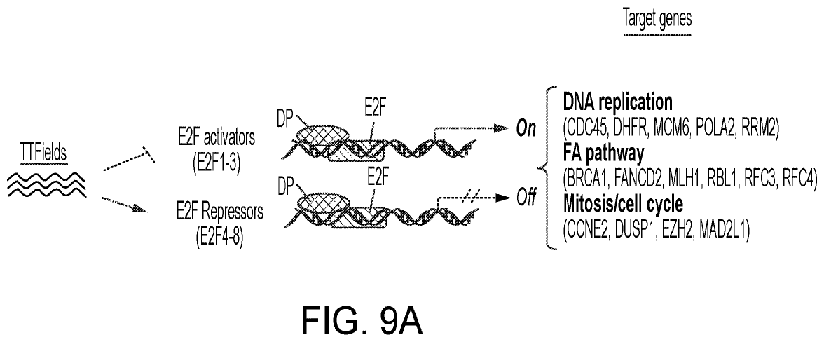

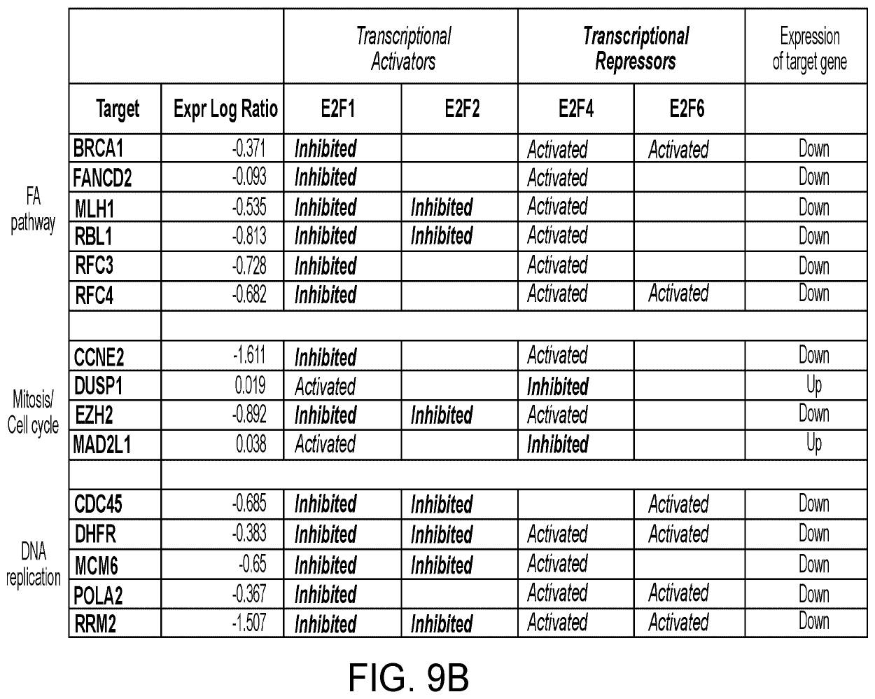

FIGS. 9A-9B illustrate the exemplary activity of transcriptional activators (E2F1, E2F2) and transcriptional repressors (E2F4, E2F6) with respect to the indicated target genes in the FA Pathway (BRCA1, FANCD2, MLH1, RBL1, RFC3, RFC4), Mitosis/Cell Cycle (CCNE2, DUSP1, EZH2, MAD2L1), and DNA Replication (CDC45, DHFR, MCM6, POLA2, RRM2). Taken together, the data in FIGS. 9A-9B show that TTFields inhibit transcriptional activators (E2F1, E2F2) and activate transcriptional repressors (E2F4, E2F6) for these three classes of genes.

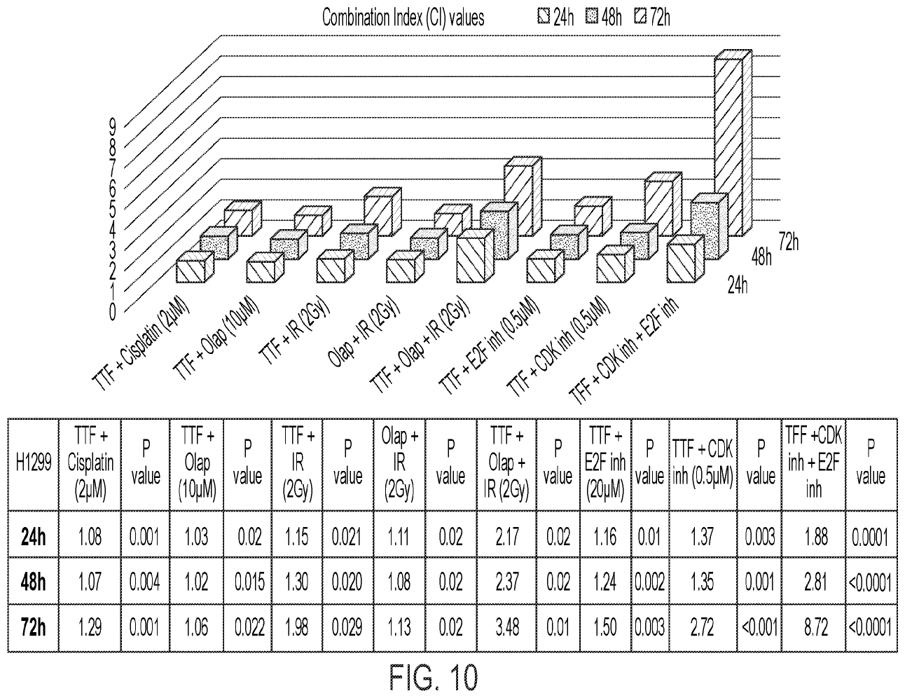

FIG. 10 provides exemplary combination index (CI) data for various combinations of TTFields and drugs after 24, 48, and 72 hour exposure to TTFields in H1299 cells. Combination index studies are used to determine the additive effect or synergism of the biological effects of drug combinations. See, e.g., Chou et al., Drug combination studies and their synergy quantification using the Chou-Talalay method, Cancer Res, 2010 Jan. 15; 70(2):440-6. In one aspect, the combination of TTFields exposure for 72 hours, 20 μM of E2F inhibitor HLM006474 and 0.5 μM of abemaciclib resulted in an unexpected and surprisingly high combination index of 8.72.

### Example 1—Experimental Materials and Methods

Cell Culture

Human NSCLC cell lines (H157, H4006, A549, and H1299) were purchased from American Tissue Culture Collection. All these cell lines were grown in RPMI medium supplemented with 10% (v/v) fetal bovine serum (Atlanta Biologicals, Flowery Branch, Ga., USA) and penicillin/streptavidin (final concentration 50 μg/ml; Sigma-Aldrich, St. Louis, Mo., U29SA). All cells were grown at 37° C. in a humidified incubator constantly supplied with 5% CO₂.

Tumor Treating Fields

The inovitro system (NovoCure Ltd, Haifa, Israel) was used to generate TTFields that use two pairs of electrodes printed perpendicularly on the outer walls of a Petri dish composed of high dielectric constant ceramic (lead magnesium niobate-lead titanite (PMN-PT)). The transducer arrays were connected to a sinusoidal waveform generator that generate low-intensity electric fields at the desired frequencies in the medium. The orientation of the TTFields was switched 90° every 1 second, thus covering the majority of the orientation axis of cell divisions. Plate temperature was maintained at 37° C. by placing the plates in a refrigerated incubator where the temperature was maintained at 19° C. to dissipate the heat generated by the inovitro system. The temperature was measured by 2 thermistors (Omega Engineering, Stamford, Conn., USA) attached to the ceramic walls. All cell suspensions were grown on a cover slip inside the inovitro dish (NovoCure Ltd) and treated with TTFields for the times indicated in the Figures.

Cell Growth Assay

Human NSCLC (H157, H4006, A549, and H1299) cell lines were treated with different frequencies of TTFields indicated for 24, 48 and 72 hours, and cell growth was counted using a Beckman coulter counter (Beckman Coulter Inc, Indianapolis, hid., USA) in triplicates for each sample. Growth curve graphs were drawn using the average cell number counted at each time point and the given TTFields frequency using GraphPad Prism V.6 (GraphPad Software Inc, La Jolla, Calif, USA).

Cell Cycle Analysis

Cells at specific times and treatments were harvested and fixed in 75% ice-cold ethanol at −20° C. for 24 hours. Fixed cells were washed with PBS and incubated in 500 μl of PI staining solution, that is, PBS containing 1 mg/ml RNAse A (Sigma-Aldrich), 0.05% triton X-100 and 30 μg/ml of PI (Sigma-Aldrich) for 30 min at 37° C. The cell cycle distribution was determined using a FACSCalibur system (BD Biosciences, San Jose, Calif., USA). More than 10,000 cells per sample were counted and the results were analyzed using FlowJo software v8.7.1 (Tree Star Inc, Ashland, Oreg., USA).

Labeling and Hybridization of RNA for Gene Expression Analysis

Illumina Whole Genome HumanWG6 v4 Expression BeadChips (Illumina Inc, San Diego, Calif., USA) were used. Each RNA sample (0.5 μg) was amplified using the Illumina TotalPrep RNA amplification kit with biotin UTP (Enzo Life Sciences, Inc., Farmingdale, N.Y., USA) labeling. T7 oligo(dT) primers were used to generate single-stranded cDNA followed by a second-strand synthesis to generate double-stranded cDNA, which is then column-purified. In vitro transcription was done to synthesize biotin-labeled cRNA using T7 RNA polymerase. The cRNA was then column-purified and checked for size and yield using the Bio-Rad Experion system (Bio-Rad Laboratories, Hercules, Calif, USA). cRNA (1.5 μg) was then hybridized for each array using standard Illumina protocols with streptavidin-Cy3 (Amersham Biosciences, Piscataway, N.J., USA) being used for detection. Slides were scanned on an Illumina Beadstation (Illumina Inc).

Data Processing and Significance Analysis of Differential Gene Expression

Summarized expression values for each probe set were generated using BeadStudio 3.1 (Illumina Inc). The data were background-subtracted and quantile-quantile-normalized across samples using the MBCB algorithm. Normalized gene expression values were used to generate plots for comparisons. Analysis of differentially expressed genes in treated cell lines was performed using SAM. FDR<0.05 was considered to be statistically significant. Clustering analysis and heatmaps were generated using the Partek Genomic Suite software (Partek Incorporated, St. Louis, Mo., USA). Gene ontology and pathway analysis was performed using IPA (QIAGEN, Redwood City, Calif., USA).

Immunoblotting

Laemmli sample buffer (4×; Bio-Rad Laboratories) was added to 30 μg of each protein sample and the mixtures were boiled at 95° C. for 10 min. Protein mixtures were then loaded on 10% SDS-PAGE gel followed by transfer to PVDF membrane for 1 hour at 90 V at 4° C. The membrane was blocked with 5% fat-free milk in PB ST for 1 h at room temperature and probed with anti (3-actin (1:5000; Cell Signaling, Danvers, Mass., USA), anti-BRCA1 (1:1000), anti-FANCD2 (1:2000) and anti-FANCA (1:500; Novus Biologicals LLC, Littleton, Colo., USA) in PBST containing 2% bovine serum albumin (Thermo Fisher Scientific Inc, Bridgewater, N.J., USA) overnight at 4° C. Membranes were washed with phosphate-buffered saline with 0.1% Tween-20 (TBST; 3×10 min, each) followed by incubation with secondary antibodies (I:5000) conjugated with horseradish peroxidase (GE Healthcare, Buckinghamshire, UK) for 1 hour at room temperature. Membranes were developed using a chemiluminescence detection kit (Thermo Scientific, Rockford, Ill., USA) on FluorChem M system (ProteinSimple, San Jose, Calif, USA). Quantification was done using the ImageJ software (NIH, Bethesda, Md., USA) and normalized using the corresponding actin density.

Immunofluorescence

Cells were seeded on glass coverslips and after treatment cells were washed and fixed with ice-cold methanol. The samples were blocked with 10% normal goat serum for 1 hour and incubated with phospho-histone-γ-H2AX antibody (Ser139; Upstate Biotechnology, Temecula, Calif, USA) and p53-binding protein 1 (53BP1) antibody (Cell Signaling). Samples were washed three times for 5 min in PBS, and then incubated with Alexa Fluor 488-conjugated antirabbit antibody and Alexa Fluor 555-conjugated anti-mouse antibody (Invitrogen, Carlsbad, Calif, USA) for 1 h. Nuclei were counterstained with DAPI contained in Vecatshield mounting medium (Vector Laboratories Inc, Burlingame, Calif, USA). The stained cells were then analyzed under a fluorescence microscope (Axio Imager M2, Carl Zeiss, Thornwood, N.Y., USA) with a×63 objective (oil immersion, aperture 1.3) with five slices of z-stacks of 0.2 μM thickness each. Quantitative image analysis of 40 nuclei from each experiment was performed using Cell module in Imaris software version 8.0 (Bitplane, Concord, Mass., USA).

Radiation Exposure and Clonogenic Cell Survival

To study the effect of radiation sensitivity on NSCLC cells, exponentially growing cells were treated with IR using a Mark II Cs irradiator (J L Shepherd and Associates) at a dose rate of 3.47 Gy/min, followed by immediate application of TTFields for 24, 48 and 72 hours. Cells were then re-seeded into 60 mm dishes and incubated for up to 2 weeks. Colonies containing 50 or more cells were considered viable. The data are presented as the mean±S.E.M. of three independent experiments. The radiosensitization effect of TTFields was evaluated according to The Highest Single Agent approach by calculating the CI as given below.

CI=(SF_{IR}×SF_{TTFields})/SF_{IR}+_{TTFields} where SF=Survival fraction

The combination effect was considered enhanced/synergistic when CI>1, additive when CI=1. Statistical significance for a positive effect was determined by the P-value of a two-way ANOVA multiple comparison statistical test comparing the combination (TTFields plus IR) to the single agent showing the greatest cell killing for a given dose and time after IR.

### Example 2—Proteometric Analysis of Lung Cancer Cells Exposed to TTFields

In some aspects, proteomic analysis of lung cancer cells exposed to Tumor Treating Fields identified that the dysregulation of the E2F-Rb-CDK4/6 axis rendered tumor cells susceptible to described novel combination therapies that target CDK4/6 and/or E2F. One mechanism described for TTFields induced cell death has been via the disruption of mitosis while a more recent examination suggests that TTFields causes replication stress, and down-regulates DNA repair and cell cycle checkpoint genes. However, the exact cause of the downregulation of DNA repair and cell cycle checkpoint genes has been elusive. To that end, the disclosed techniques employed relative quantitative proteomic analysis using tandem mass tags (TMT). All samples underwent trypsin digestion and labelling with different TMT reagents. They were then combined, and the mixture was processed on an Orbitrap Fusion mass spectrometry device.

Peptide quantitation was accomplished by comparing the intensities of the TMT reporter ions. STRING DB analysis of differentially expressed proteins revealed interaction networks that included cell cycle, DNA damage repair and replication, and transcriptional and translational regulation. Upstream analysis of key genes associated with cell cycle checkpoint and DNA repair identified reduced expression of the transcriptional activators E2F1 and E2F2 and increased expression of the transcriptional repressor F2F6, suggesting that TTFields affects the CDK-RB-E2F axis. For example, the downregulation of key DNA repair genes including RAD51, BRCA1 and BRCA2 could be explained, for example, through the upregulation of the transcriptional repressors E2F4 and E2F6 (a known repressor of BRCA1).

These proteins are involved in homologous recombination repair and nucleotide excision repair, but also with replication fork maintenance, replication fork collapse and overall replication stress, the latter of which likely leads to cell death. Therefore, in one example, TTFields was combined with the E2F inhibitor HLM006474 with or without the CDK4/6 inhibitor abemaciclib. TTFields in combination with either inhibitor enhanced cell killing synergistically, as compared to TTFields alone, while the triple combination was found to be highly lethal (>90% by 72 hours) as measured by clonogenic assay followed by the Highest Single Agent approach to determine synergy. Taken together, in one example, the results identified the CDK-RB-E2F axis as a novel druggable target that can be used in combination with TTFields for cancer therapy.

### Example 3—Further Genomics and Proteomics Support for Combination Therapy Including TTFields

A transcriptomics approach was employed by research groups in the past in order to capture the whole picture of biological processes in a non-targeted and un-biased manner and understand global gene expression changes upon TTFields exposure in a series of non-small cell lung cancer (NSCLC) cells. Ingenuity Pathway Analysis (IPA) results of TTFields responsive genes suggested that alterations occurred in cell cycle and mitotic regulatory pathways, which is consistent with previous studies but also revealed a significantly downregulated BRCA1 DNA-damage response pathway (P<0.05) with TTFields exposure. However, exactly what causes the downregulation of DNA repair and cell cycle checkpoint genes had been elusive.

Accordingly, experiments were performed including proteomics analysis in order to explore TTFields induced proteome level changes in order to determine how genomics level expression changes are translated into protein expression changes, the functional players for biological activity. Relative quantitative proteomic analysis using tandem mass tags (TMT) was used to quantify the proteomes of control and TTFields treated conditions in H1299 cells. There were total 106 and 541 differentially expressed proteins at 24 hours and 48 hours respectively after TTFields exposure. STRING database analysis of differentially expressed proteins revealed interaction networks that included cell cycle, DNA damage repair and replication, mitochondrial dysfunction and transcriptional and translational regulation. The same patterns of expression changes for members of the FA pathway, cell cycle and DNA damage and replication pathways were observed at the protein level from proteomics as well as at the mRNA level from transcriptomics analysis, as is illustrated in FIGS. 8A-8F.

FIGS. 8A-8F illustrates temporal mRNA level expression changes from transcriptomics data, protein level expression changes from proteomics data and validation of protein level changes by western blot for some of the FA pathway members identified as downregulated suggests a similar expression pattern for FA pathway members at both the gene and protein levels.

Additionally, experiments were performed to identify the key upstream regulatory network mechanism(s) which act as a control node for the expression of several downstream pathways such as cell cycle, DNA damage repair and replication etc., because these regulatory nodes exert maximal control over dysregulated pathways but minimal control over unperturbed pathways. IPA Upstream Regulator Analytic analysis of TTFields induced differentially expressed proteins identified upstream transcriptional regulators such as reduced expression of E2F1 and E2F2 and increased expression of E2F6 which are transcriptional activators and repressors respectively, suggesting that TTFields affects the CDK-Rb-E2F axis and dysregulates key DNA repair proteins including RAD51, BRCA1 and BRCA2 through E2F4 and E2F6 signaling and replication fork related (MCM6, RFC3, RFC4) mitosis related proteins (BUB3, CCNE2, EZH2) as is illustrated in FIGS. 9A-9B. These proteins are involved in cell cycle, homologous recombination repair, nucleotide excision repair, replication fork maintenance, replication fork collapse and overall replication stress.

More particularly, FIGS. 9A-9B illustrate upstream regulator analysis and quantitative E2F target expression changes from differential proteome analysis. TTFields inhibit the transcriptional activators (E2F1-3) and increase the transcriptional repressors (E2F4-8) of the E2F family of transcription factors. As such, there was a decrease in the expression of the Fanconi Anemia (BRCA1, FANCD2, MLH1, RBL1, RFC3, RFC4) and DNA replication pathway proteins (CDC45, DHFR, MCM6, POLA2, RRM2) and there was a differential impact on the mitosis/cell cycle pathway proteins (CCNE2, DUSP1, EZH2, MAD2L1) that informs us of the complexity of these activators/inhibitors of transcription on specific targets. Protein expression change ratios (Log 2) for different pathway proteins and the direction of transcriptional factors and final target protein expression are provided in the table. The upstream regulator analysis is based on prior knowledge of expected effects between transcriptional regulators and their target genes stored in the Ingenuity® Knowledge Base and examines how many known targets of each transcription regulator are present in the user's dataset, and also compares their direction of change (i.e., expression in the TTFields sample(s) relative to Control).

A comparative analysis of transcriptome and proteome level changes was performed which indicated that the CDK-Rb-E2F axis acts as an upstream regulatory node for the effects observed with TTFields exposure which are implicated in cell cycle, DNA damage repair and replication stress pathways. The CDK-Rb-E2F axis is druggable, indeed it is now a major drug target in cancer treatment, and in doing so, targeting this axis would change how TTFields could be used going forward including becoming an integral therapy that would enhance conventional radiation and chemotherapies that target DNA repair, cell cycle checkpoint or proliferation and survival pathways.

FIG. 10 provides comparison of combination index values for different agents and ionizing radiation (IR) together with TTFields. In one example, the results suggest that blocking the CDK-E2F-RB axis using both a CDK inhibitor and E2F inhibitor in combination with TTFields is highly effective compared to individual agents or other agents. P values for each combination are provided in the table.

The systems and methods described herein may use TTFields in combination with agents after primary therapies are applied. By combining TTFields with RB-E2F axis agents, integral therapies may enhance downstream therapies such as radiation, chemotherapy or other therapies that target DNA repair, cell cycle checkpoint, or proliferation and survival pathways.

The systems and methods described herein can be used for the treatment and/or mitigation of gliobastoma, mesothelioma, pancreatic cancer, lung cancer, ovarian cancer, cervical cancer, prostate cancer, skin cancer, peritoneal cancer and the like. The systems and methods herein may be used to improve upon conventional systems that apply TTFields by combination therapeutics.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the claims listed below, and equivalents thereof.

1. A method of reducing survival of cancer cells in a subject, comprising:
administering an E2F inhibitor and a CDK4/6 inhibitor to the subject such that the cancer cells are delivered a therapeutically effective concentration of the E2F inhibitor and a therapeutically effective concentration of the CDK4/6 inhibitor, wherein the CDK4/6 inhibitor comprises at least one of palbociclib, ribociclib, or abemaciclib; and
applying alternating electric fields to the cancer cells at a frequency between 80 and 300 kHz.

2. The method of claim 1, wherein at least a portion of the applying is performed simultaneously with at least a portion of the delivering.

3. The method of claim 1, wherein the applying has a duration of at least 72 hours.

4. The method of claim 1, wherein the frequency of the alternating electric field is between 100 and 200 kHz.

5. The method of claim 1, wherein the alternating electric field has a field strength of at least 1 V/cm in at least some of the cancer cells.

6. The method of claim 1, wherein the therapeutically effective concentration of the E2F inhibitor in the cancer cells is from about 10 μM to about 50 μM.

7. The method of claim 1, wherein the therapeutically effective concentration of the E2F inhibitor in the cancer cells is from about 20 μM to about 40 μM.

8. The method of claim 1, wherein the therapeutically effective concentration of the CDK4/6 inhibitor in the cancer cells is from about 0.1 μM to about 5 μM.

9. The method of claim 1, wherein the therapeutically effective concentration of the CDK4/6 inhibitor in the cancer cells is from about 0.5 μM to about 2 μM.

10. The method of claim 1, wherein the E2F inhibitor comprises HLM006474.

11-13. (canceled)

14. The method of claim 1, wherein the CDK4/6 inhibitor is abemaciclib.

15. The method of claim 1, wherein the E2F inhibitor is HLM006474 and the CDK4/6 inhibitor is abemaciclib.

16. The method of claim 1, wherein the cancer cells are selected from the group consisting of lung cancer cells, breast cancer cells, pancreatic cancer cells, glioblastoma cells, prostate cancer cells, liver cancer cells, fallopian tube cancer cells, peritoneal cancer cells, skin cancer cells, liver cancer cells, and ovarian cancer cells.

17. The method of claim 1, wherein the survival of the cancer cells is reduced by 20 to 100 fold compared to cancer cells that do not receive exposure to alternating electric fields and do not receive delivery of an E2F inhibitor and a CDK4/6 inhibitor.

18. A method of killing cancer cells in a subject, comprising:
administering an E2F inhibitor and a CDK4/6 inhibitor to the subject such that the cancer cells are delivered a therapeutically effective concentration of the E2F inhibitor and a therapeutically effective concentration of the CDK4/6 inhibitor, wherein the CDK4/6 inhibitor comprises at least one of palbociclib, ribociclib, or abemaciclib; and
applying alternating electric fields to the cancer cells at a frequency between 80 and 300 kHz.

19. The method of claim 18, wherein at least a portion of the applying is performed simultaneously with at least a portion of the delivering.

20. The method of claim 18, wherein the applying step has a duration of at least 72 hours.

21. The method of claim 18, wherein the E2F inhibitor comprises HLM006474.

22-23. (canceled)

24. The method of claim 18, wherein the CDK4/6 inhibitor is abemaciclib.

25. The method of claim 18, wherein the E2F inhibitor is HLM006474 and the CDK4/6 inhibitor is abemaciclib.

26. The method of claim 18, wherein the cancer cells comprise defects in a BRCA pathway.

27. The method of claim 18, wherein the cancer cells are selected from the group consisting of lung cancer cells, breast cancer cells, pancreatic cancer cells, glioblastoma cells, prostate cancer cells, liver cancer cells, fallopian tube cancer cells, peritoneal cancer cells, skin cancer cells, and ovarian cancer cells.

28. The method of claim 18, wherein the therapeutically effective concentration of the E2F inhibitor in the cancer cells is from about 10 μM to about 50 μM.

29. The method of claim 18, wherein the therapeutically effective concentration of the CDK4/6 inhibitor in the cancer cells is from about 0.1 μM to about 5 μM.
